(19) United States
(12) Patent Application Publication
(10) Pub. No.: US 2021/0401773 A1
(43) Pub. Date: Dec. 30, 2021

(54) ye-COVIDICIN Botanical and Traditional Medicine to Treatment for COVID-19

(71) Applicant: Yvon Etienne, Coral Springs, FL (US)

(72) Inventor: Yvon Etienne, Coral Springs, FL (US)

(21) Appl. No.: 16/857,479

(22) Filed: Jun. 25, 2020

(51) Int. Cl. A61K 31/122; A61M 16/10; A61K 36/752; A61F 7/08

(52) U.S. Cl. CPC A61K 31/122; A61F 7/08; A61K 36/752; A61M 16/109

(57) ABSTRACT

The main objective of this study was to evaluate the effects of heat, Momordicilin and antibiotics on Coronavirus. This study selected 12 individuals tested positive for COVID-19 which included two healthcare providers, one friend, a Massachusetts' family, and six family members in Florida including the inventor. All individuals were testing positive for COVID-19 excluding two individuals and inventor who didn't test. They had fever, mild headache, chest pain, cough and loss of appetite. All individuals were taking facial steaming(vapor) between 5-15 minutes for four consecutive days, a cup of Cerasee Tea with lime/lemon(Extracted-Momordicilin) three times a day for seven days and with or without any antibiotic as prescribed by their physicians. All patients felt relief immediately after taking the first dose of heat and Asosi Tea or antibiotic. All symptoms were gone in five days. After seven days, all individuals had no symptoms and re-tested. The results came negative for COVID-19. These results indicate that combination of heat, momordicilin and with or without antibiotic treat COVID-19.

### SPECIFICATION FOR ye-COVIDICIN

ye-COVIDICIN is an alternative medicine that treats COIVID-19. Alternative medicine which is a combination of medicinal plant and traditional medicine all together to treat specific illnesses. ye-COVIDICIN treatment for COVID-19 is comprising of combination of specific heat process which is facial steaming, momordicilin extracted from Asosi Tea with lime and with or without any antibiotic if not allergic. Asosi or Cerasee leaves possesses specific anti-viral agent can target any flu viruses including HIV and it is also a detoxed agent for blood purification as well as it can boost the human immune system due to possess lots of phytochemicals, cytotoxic agent and vitamins. The first step in taking ye-COVIDICIN is the extraction of momordicilin which is Asosi or Cerasee Tea with lime or lemon. Take 3 grams of Asosi and boil it with 2 cups of drinking water in a cleaned casserole for 15 minutes FIG. 7(a). When the Asosi Tea is ready, added half lime or lemon in the warm tea to create or extract the momordicilin which is a powerful antiviral agent can attack COVID-19 FIG. 7(c).

Viruses are non-living cells which depend on host cell to replicate which make them extremely difficult to identify any specific anti-viral agents to target or inhibit the viral proteins in those viruses. The inventor realizes that heat can denature proteins as well as the virulent protein in COVID-19. The inventor comes up with a widely used empirical and medieval medicine which is vapor (facial steaming). This specific heat process is the most important and the second step in ye-COVIDICIN. Put 2 litters of water on a pot FIG. 1. When the water is boiled, put a towel on your head, and directly stand up 42 cm from your head to the stove to take the vapor between 5-15 minutes FIG. 2. Dry your body when getting sweated. Finally, after taking the specific heat process, drink a cup of warm Asosi Tea with lime or lemon with any antibiotic or without any antibiotic. You will feel immediately relief. Continue taking the heat process for four days. Drink a cup of Cerasee Tea with lime twice or three times a day with or without antibiotic for seven days. All symptoms will be gone in five days and patient should be cured and re-tested negative for COVID-19. These results indicate that combination of specific heat process, momordicilin and with or without antibiotic treat COVID-19 or inhibit Coronavirus. This specific alternative medicine has been used in human and provided the intended results. Therefore, ye-COVIDICIN has all the scientific principles requirement to be claimed as treatment for COVID-19. Furthermore, Alternative medicine is the future in combating novel viruses which is barred from others to use “momordicilin” and “heat or heat process” for development of any vaccine or drug for the Coronaviruses and any other flu viruses for the duration of the patent's term. The following listing of specifications A and B below serve to define possible uses or applications for the ye-COVIDICIN.

### A. SPECIFICATION FOR THE HEAT PROCESS (FACIAL STEAMING)

1) Put ¹/₂ gallon of water or any desired quantity of water on a pot or container on the stove
2) Boil the water (FIG. 1)
3) Get a towel ready when the water is boiled
4) Lower stove temperature to number 5 or 6 when the water starts boiling to avoid burning due to high steam. Be extremely careful with this process or use Thermophore Heater.
5) Put the towel on your head and stand up above the pot with boiled water from a distance from 21 centimeters or 42 cm to the stove or 1 ⅓ ft. on the vapor about 5-15 minutes. This will allow the vapor to directly hit to your face and nose to injure COVID-19 (FIG. 2)
6) Dry your body or face with the towel when you sweated. This heat process especially is very tough when you have COVID-19 Virus or flu, but you will feel a better instantly
7) Take the vapor for 5-15 minutes. Then, you might feel that you can breathe much better immediately while taking the vapor on the first day. The headache will be gone, but the fever or cough won't go away until the fourth day of the doses. Don't worry! However, you might feel a chest pain when you cough or sneeze after you finish taking the first heat process if you were experiencing severe breathing problem due to COVID-19
8) Repeat the heat process (facial steaming) consecutively for four days
9) After taking the heat process each time, drink the Asosi Tea and lime with or without any antibiotic if not allergic as prescriber by your healthcare provider to hydrate the body and to start inhibiting the COVID-19
Or Warm wet towel on enfant (0-2) or Thermophore Heat Pads is recommended for kids 3-year-old up to 12 years old under parental surveyance (FIG. 3). See Thermophore Specs.
Remove the water in the warm towel for enfants. Gently place the warm towel or heat pat on kid's chest with clothes or front between 3-10 minutes to get the heat process (FIG. 4)
*Highly Important-Please avoid drinking all cold drinks and stay inside and rest your house during treatment as much you can at least 3 days. Please wear a jacket when opening the refrigerator or freezer. Unless the temperature is reached 80° F., please stay inside and rest as much possible unless you need foods or medical supplies or emergency.
*House or room must be decontaminated with bleach and heat (Specific Process) to kill COVID-19
*(Boiled water with leaves. Pour them into a container to keep the vapor longer in the developing countries. The patient seat directly in front of boiled water with a towel to get the heat process (FIG. 5) for 20 minutes)

### B. EFFECTS OF THE HEAT PROCESS ON COVID-19

This special heat process which is facial steaming is very important process in denaturing the COVID-19 proteins so that the virus can be injured or killed. The human body is around 60 percent water and water is spreading out in different areas in our body. Taking the vapor for 5-15 minutes in your face will cause the temperature of the water in our body is hot or gradually increased. Then, the body begins tapping different water reserves from places like the blood stream, fat, muscles and kidneys to maintain our normal bodily functions where the COVID-19 is attacking. The virulent protein in the COVID-19 will start injuring by the heat. When external environments are exceedingly warm, the heat is produced inside the body is typically transported to the blood. The blood then carries the heat through numerous capillaries that are located directly under the skin. Our hypothalamus works as a thermostat to regulate the body temperature due to homeostasis process that stimulates the thermoregulation. It will be responding to internal and external stimuli and making adjustments to keep the body cool down or normal to allow all system orgasms to adequately function during or after heat process.

The hypothalamus will release chemicals and hormones related to temperature to allow the blood to have more oxygen. The heat activates sweat glands; these glands bring that water along with the body's salt to the surface of the skin as sweat. The body will start sweating seeps through our pores and evaporates and taking heat away from our body. Sweat is also a means by which the body cools itself down due to the homeostasis process to maintain core body temperature. This is another reason why it is very important to stay hydrated during the heat process-not only to maintain adequate intravascular fluid volume, but also to aid in conduction processes that cool the body down. High risks or immuno-compromised individuals should not take the heat process for too long due to decreased cardiac function is also a notable risk factor for abnormal thermoregulatory function. It is advisory for immuno-compromised individuals to take the heat process via warm towel or Thermophore for duration of 5-20 minutes to injure the virus.

Moreover, this special heat process will allow the hypothalamus to respond COVID-19 Infectious organisms by releasing fever-producing chemicals that change body temperature. When COVID-19 Virus will expose to the heat, the virus will start depressed and cannot continue to release the viral genes or particles to infect new host cells and the COVED-19 Virus will not be able to replicate in the body host cells. This heat process will start strengthening the body's immune system to combat that invader COVID-19. The body will need to be hydrated by warm Asosi Tea with lime to avoid dehydration, dizziness, weakness excessive chest pain and confusion and to injure the COVID-19. When the special heat process and Asosi Tea with lime are repeating twice, the momordicin from the Asosi Tea will attack the virus and allow the body antibodies to develop antiviral enzymes to be activated to fight the infection further. The infected body will start detoxing and the virus will be washed out during urination with continuation of taking the Asosi Tea twice or three times a day for 7 days. Finally, COVID-19 will be injured or inhibited.

### C. EXTRACTION OF MOMORDICILIN (ANTIVIRAL) OR (CERASEE TEA+LIME)

1) Extract momordicilin from plant based—Formation of Momordicilin from Asosi Leaves
I. Find or buy some fresh or dried Cerasee\Asosi leaves (Found in Florida or California . . .)
II. Formation of Momordicilin from Asosi
   a. Take 2 or 3 cups of drinking water
   b. Pour the drinking water in a cleaned casserole or pot
   c. Boil the water in stove or microwave or others. When the water is boiled, add about 2 g dried Asosi or fresh Asosi leaves (1 garlic slide can be added if desired) to boil for 8-15 minutes. The Asosi Tea's color will be green (FIG. 7(a))
   d. Strain the Asosi Tea in a cup to remove any byproducts or leaves (FIG. 7 (b))
   e. Squeeze ½ lime or lemon in the tea to create the antiviral medicine (Asosi Tea will turn white hydrated crystalline in present of acid citric) (FIG. 7(c))
   f. Drink the warm Asosi Tea twice or 3 times\day for a consecutive 6 or 7 days
   g. Take any antiviral or antibiotic or OTC Theraflu if not allergic as prescribed by your Health Care Provider (physician) for much faster recovery if available
   h. COVID-19 virulent cells will be dead or injured in the cells host after 7 days and all symptoms must be stopped. Also, the chest pain or fever should be gone in 5 days. Then, the Asosi Tea will detox or decontaminate the human body through urination. You will be freed from COVID-19. Please wash hand with warm water with soap for 30 seconds and wear mask when going to stores or work to prevent re-catching the COVID-19 Virus again.
*House or room must be decontaminated with bleach and heat to kill COVID-19 virulent germs in the air because when breathing, the COVID-19 germs can stay in suspension up to 16 hours waiting for a host cell to duplicate. Clean all doors, countertop, bathroom with bleach or tiled floor with bleach to kill any germs. Wear mask and leave the house for 2-3 hours for the bleach to kill the COVID-19 germs inside the house. Then, turn on the heater if room temperature is not 82° F. for 2 hours or open the window if it is hot. Turning on A/C after decontamination process.
*Take any antibiotic or OTC Theraflu or Tamiflu if not allergic as prescribed by your Health Care Provider (HCP) to speed up the recovery. If you don't have any antibiotic or antiviral medicine, the Asosi Tea+vapor will be enough to injure or kill the virus based on trial. Also, antibiotic+heat without Asosi can inhibit COVID-19.
*Enfant and kids can drink ⅓ cup of Asosi+Lime+Honey twice a day as well as any prescribed flu or antibiotic

### D. WHY ASOSI TEA OR MOMORDICILIN

The Asosi Tea with lime or Momordicilin has two main properties which are to hydrate the body and to inhibit or kill the virulent genes in the body host cells. When patients are dehydrated during heat process, they contain a decreased amount of intravascular volume. There are not enough fluids in the body and there also is not enough blood to circulate in those veins. Heat will remain retained, blood will become thicker, and the heart becomes strained during heat stress. It cannot fully support their respiratory, hepatic, renal, and circulatory systems, but this specific heat process does the opposite. Momordicin or Asosi leaf or fruit tea has enzyme with both cytostatic and cytotoxic properties that can inactivate the viral infection or destroy infected virus cells and can hydrate the body to function properly as well as boost the hypothalamus to regulate the body temperature,. Moreover, the Asosi will purify the blood. See Fresh Asosi Leaves Picture.

### E. FIGURES

FIG. 1 shows that patient should put ½ gallon of water or any desired quantity of water on a pot or container on the stove to boil. FIG.1 also shows the patient lowers stove temperature to number 5 or 6 when the water starts boiling to avoid burning due to high steam and put the towel on your head and stand up above the pot with boiled water from a distance from 21 centimeters or 42 cm to the stove or 1 ⅓ ft. on the vapor about 5-15 minutes. This will allow the vapor to directly hit the face and nose of the patient to injure or denature the COVID-19 viral protein. FIG. 2. In addition, FIG. 3(a) and FIG. 3(b) show that Thermophore Heater Pad has been placed on chest and front head of kid under parental supervision. Moreover, FIG. 4(a) and FIG. 4(b) have shown that warm towel is smoothly placed on the chest and front head of enfant. Additionally, FIG. 5(a) and FIG. 5(b) show that 1 gallon of water and any leaves were added in a pot and boiled them 20 minutes on wood fire. When the water is boiled, pour the boiled water and leaves into a container and sit down in front of the boiled water to take the vapor (facial steaming) for 20 minutes. FIG.6 has shown the chemical equation of extraction of momordicilin from Cerasee leaves. Finally, FIG. 7(a), FIG. 7(b) and FIG. 7(c) show that the preparation of momordicilin by adding 2 cups of drinking water into a cleaned casserole or pot. Then, boiled with 2 g dried or fresh Asosi for 8-15 minutes until the Asosi Tea becomes green. Strain the Cerasee Tea to remove any byproducts. Squeezed ½ lime or lemon into the tea to create the antiviral medicine (white hydrated crystalline is the hydrated momordicilin).

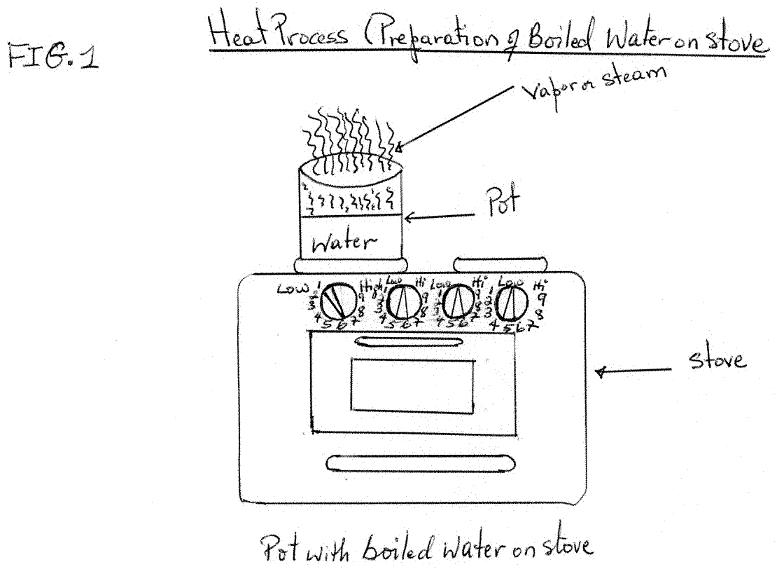

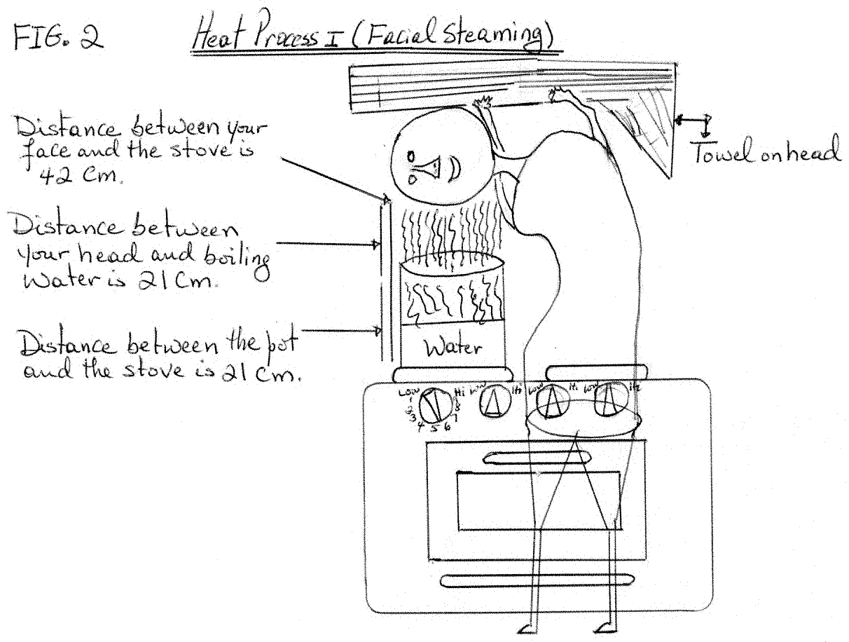

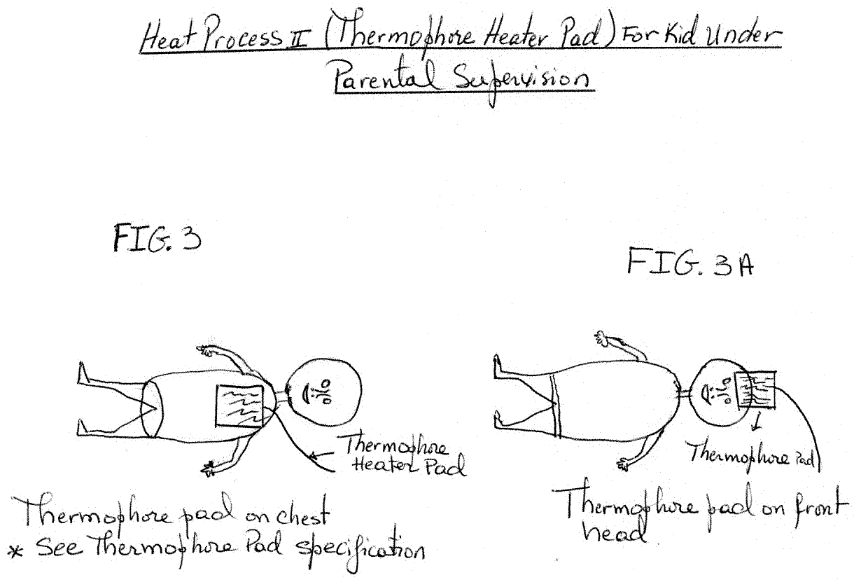

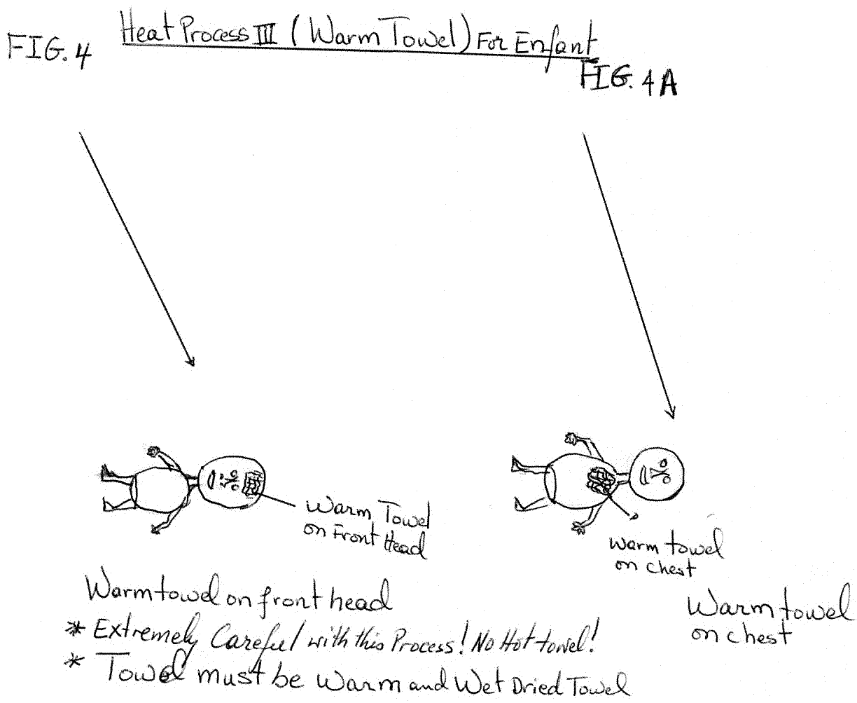

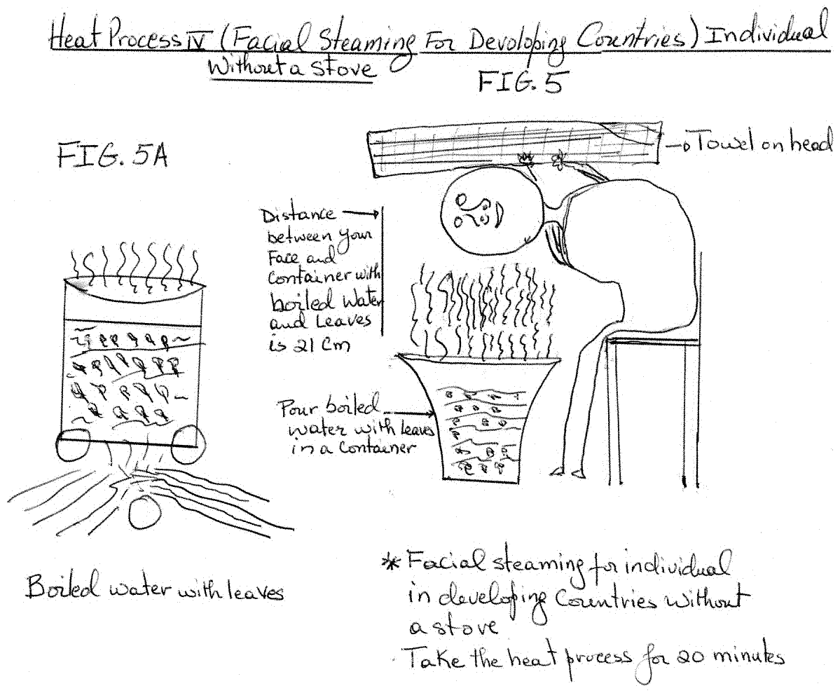

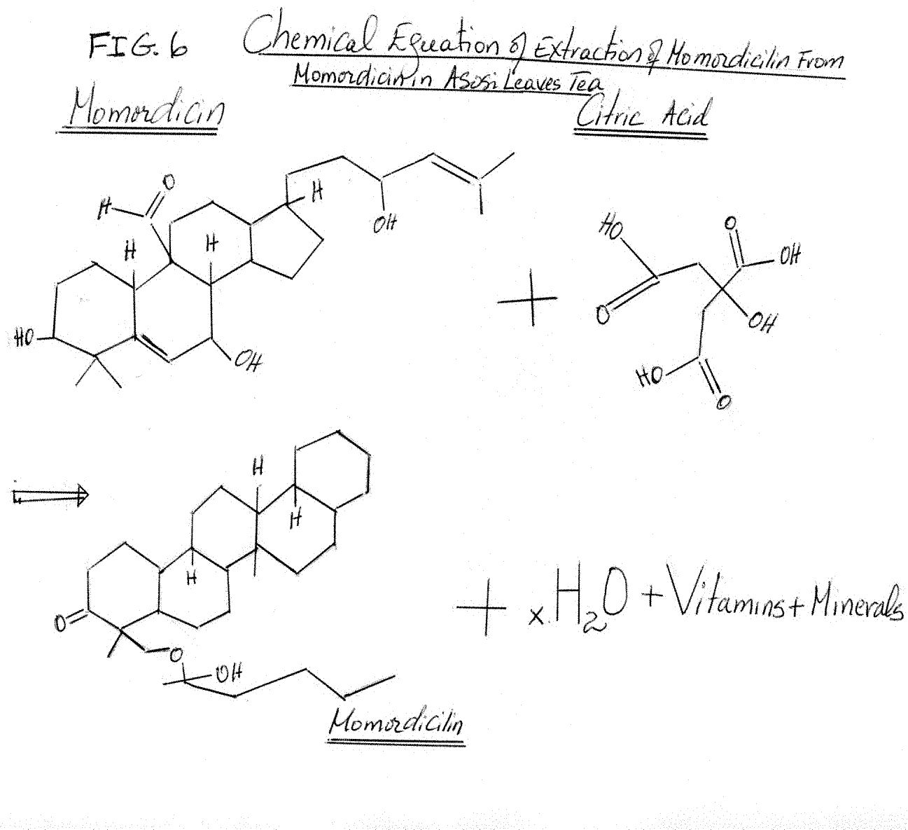

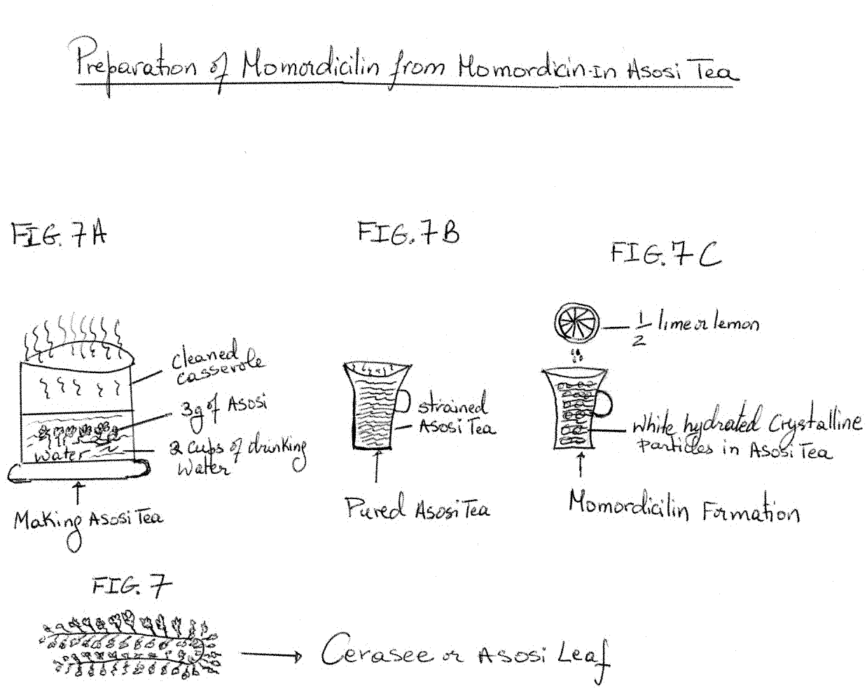

### F. DESCRIPTION: CHEMICAL FORMULA OF MOMORDICILIN

The active component of ye-COVIDICIN is momordicilin. Momordicilin is extracted from Momordicin by lime (FIG. 6). Momordicin is a chemical compound found in the leaves of Asosi or bitter melon fruits. Its IUPAC Name is 3,7-dihydroxy-17-(4-hydroxy-6-methylhept-5-en-2-yl)-4,4,13,14-tetramethyl -2,3,7,8,10,11,12,15,16,17-decahydro-1H-cyclopenta[a]phenanthrene-9-carbaldehyde₃. Momordicin/momordicilin is the white hydrated crystalline particles in the tea with molecular weight of 540.9 g/ml and the molecular formula is C₃₆H₆₀O₃. Momordicilin is soluble in warm water and it is practically insoluble in citric acid.

### G. BIOLOGICAL EFFECT

Asosi fruit and leaf is a rich source of minerals (potassium, calcium, zinc, magnesium, phosphorous and iron); vitamins (C, A, E, B1, B2, B3, and B9 as folate); and glycosides, saponins, alkaloids (Vicine), triterpenes, triterpene glycosides, proteins, momordicosides, momordicin and steroids₁. Cerasee/Asosi contains anti-diabetic or cytotoxic chemical effects. It has selective efficacy for correcting metabolic aberrations or targeting cancer cells, or have beneficial effects such as abortifacient, anthelmintic, anti-inflammatory, contraceptive, gout, kidney-stone, piles, rheumatism, galactagogic, etc. Also, it improves glucose tolerance, reduces blood glucose levels, and lowers hemoglobin and anti-human immunodeficiency virus protein (₁). Cerasee fruit and leaf are rich source of antioxidants and thus could be potentially useful against the damaging effects induced by the free radical. It also increases both hepatic and red-cell shunt enzyme glucose-6-phosphate dehydrogenase₁.

### H. CLINICAL TRIALS/STUDIES

1. One of the inventor cousins was also coughing and she had a fever and loss appetite. She had chest pain when coughing, and she had breathing problem after exposing to a store in Sunrise without mask after 6 days. She was extremely scared about dying from COVID-19. One of her cousins died from COVID-19. She suspected that she caught COVID-19. She called the inventor to inform him that she did not feel well. Then, the inventor dictated her to take the same formula (Asosi with lime+Heat) except she did not have any antibiotic. She took the heat process for 15 minutes in four consecutive days. She drank the Asosi Team with lime three times a day. After four days taking ye-COVIDICIN, she had no fever or coughing. She was feeling well after 7 days. This combination process worked.
2. The inventor girlfriend caught COVID-19 and she took Hydroxychloroquine and it did not work. She got worse. The inventor explained her how to combine traditional medicine and botanical medicine to cure patients. She took the ye-COVIDICIN which is a combination of the natural detox and anti-viral agent (Asosi with Lime)+Heat+Doxycycline Hyclate 100 mg. She continued taking the process for 7 days. The headache was gone immediately after the first dose. The fever and cough were gone in five days. In the seven (7) days of doses, she got healed very fast. She got retested. The result came negative. Ye-COVIDICIN inhibits COVID-19.
3. A registered nurse, friend of the inventor in Atlanta and another friend in Alabama caught the COVID-19. They went and stay in the hospital for 14 days. They got discharged due to overcrowded patients, but they thought that they would die from COVID-19. They called the inventor. The inventor gave them the formula. They did not have the Asosi. They took the heat process and antibiotic. This process works again.
4. A family (wife, husband, and kids) in Brockton, Mass. all were tested positive for COVID-19. They went to hospital and one got worse. A friend referred them to the inventor. The inventor gave them the ye-COVIDICIN Alternative Medicine Formula. They got the Asosi leaves from Florida, they followed ye-COVIDICIN doses. The whole family took the doses all together. After seven (7) days taking the doses, they felt great and they got retested. All results came negative. They are COVID-19 free now.
5. Five (5) family members of the inventor in Margate, Florida lived in the same household were tested positive for COVID-19. The inventor gave them ye-COVIDICIN Formula and prepared the first dose for them as well as a decontamination process to kills the virulent genes inside the house. They all took Asosi Tea+Lime and heat process doses for seven (7) days. After four days taking the doses, they did not have any fever and cough. They all got retested after 7 days and the results came negative. This process again cures COVID-19.
6. The inventor was doing research on the effects of the weathers on COVID-19 in three states (Massachusetts, Georgia and Florida) as well as the effect of wearing mask to prevent or reduce COVID-19. Moreover, the inventor intentionally exposed to patients who tested COVID-19 positive to validate his own alternative medicine discovery. After 4 days exposed to COVID-19, the inventor had a fever, mild headache, pain on all his body and loss appetite. The inventor took ye-COVIDICIN Formula (Asosi with lime+Heat+Antibiotic (Amoxicillin 875 mg)) for 7 days. All symptoms were gone in the 5^{th} day of the doses. After 7 days taking ye-COVIDICIN, the inventor was feeling very well.

### I. RESULTS

1. The patient should immediately feel relief from fever, headache and pain after the first dose of heat and Asosi Tea with lime
2. The patient should be able to breathe a little much better after first dose of heat process immediately
3. The patient should feel a mild chest pain when coughing after the first heat process if experienced breathing problem due to COVID-19. Drink the warm Asosi Tea+lime\lemon will help to relief the sinus pressure a little, but the chest pain will go away after the fourth heat process doses.
4. Fever and cough as well as all symptoms would be gone in five days after taking the ye-COVIDICIN doses
5. After seven (7) or six (6) days taking the doses, patient should be freed from COVID-19
6. After treatment if wanted, the patient can take Asosi Tea twice a week for another two weeks period to boost their immune system to fight any pathogens or viral infections.
*Any antibiotic or Tamiflu or OTC Therafiu will speed up the recovery much faster
*Honey can be used for kids because Asosi has a strong bitter taste due to momordicin
*Any OTC for Flu or Dayquil can be taken to help patients to sleep. COVID-19 is very brutal & lethal at night. It is time where the patient feels most pain, fever & coughing, etc.

### J. SUMMARY OF THE INVENTION

The object of the present invention is to provide an alternative medicine treatment for COVID-19 comprising of heat process and momordicilin (specific antiviral agent from Asosi Tea with lime) as well as with or without any antibiotic to cure or inhibits COVID-19. Any antiviral medicine or antiviral to treat or cure the COVID-19 Virus should be able to produce some heat inside the body to injure this virus. Scientifically, heat, antiviral, antibiotic, salts, acids including specific alcohol and bleaches are only ways to kill any bacteria or viruses except Mad Cow disease. This specific antiviral enzyme (Momordicin with its derivative protein momordicosides) cures COVID-19 Virus. Asosi Tea is also safe for human consumption. A further object of the present invention is to provide the first human alternative medicine for use as a therapeutic medicine for COVID-19.

### K. INTENDED USE

There are no data available for teenager. However, teenager may take ye-COVIDICIN under parental supervision to avoid burning from the heat process (facial steaming). ye-COVIDICIN intended for adults. ye-COVIDICIN was tested and studied on adults in 20s, 30s, 40s, 50s and late 50s who caught COVID-19 from February 2020 to May 2020. Results showed that ye-COVIDICIN treated COVID-19.

### L. PEDIATRIC USE

There are no data available for enfant to drink Asosi Tea with lime. However, small quantity of Asosi Tea with lime might be required for 3 years old kids and up. It is imperative to add some honey in the Asosi Tea for kids due to the bitter taste of momordicin. During trial, one 3-year old girl drank about 100 ml of Asosi Tea, there was no side effect. Asosi tea is considered safe for consumption.

### M. PREGNANCY

There are no data available for early pregnant woman who drinks Asosi Tea. However, early pregnant women are discouraged to take Asosi Tea to avoid miscarriage. Late pregnancy (after 6 months) woman may drink moderate doses of Asosi Tea with lime. Trial result showed that there were no side effects on a late pregnant woman who tried some Asosi Tea. More research or studies are needed to determine the safest doses for pregnant women. But moderate heat process in the front head and antibiotic will be much preferred for pregnancy women.

### N. IMMUNO-COMPROMISED AND MENTAL HEALTH PATIENTS

There are no data available for immuno-compromised and mental health patients.

### O. DOSAGE

Adult and teenager can drink a cup (10 oz±300 ml) of warm Asosi Tea+Lime/Lemon three times a day with or without any antibiotic or Theraflu if not allergic as prescribed by HCP for seven (7) days. Kid and enfant can drink ⅓ cup (3.8 oz±100 ml) of cold Asosi Tea+Lime/Lemon+Honey as well as any flu medicine or antibiotic prescribed by their HCP twice day for 6 days. In addition, adult and teenager can take the heat process once a day between 5-15 minutes for four days. A warm wet towel treatment might be recommended twice a day for enfant for three days (No studies available). Warm wet towel or Thermophore is recommended for kids once a day for 3-12 minutes for 4 days. Moreover, mental health and high risks individuals are recommended to use the Thermophore Heater under surveyance instead of the vapor process to avoid burning.

### P. SIDES EFFECTS

Taking ye-COVIDICIN has not side effects during trials on all patients tested COVID-19 positive. It is safe to drink Asosi Tea if an individual is not allergic to it. During trials, a late pregnant woman tried some Asosi tea once a week and she had no side effects as well as a 3-year old girl drank a small quantity of Asosi Tea with honey and she had not side effects. However, early pregnant women will not be encouraged to drink Asosi Tea to avoid miscarriage. More research or studies are needed to determine the safest doses for pregnant women. Moreover, woman period cycle might or might not be shorten for five days only for one month during treatment due to detoxification agent in Asosi Tea.

### Q. OVERDOSAGE

Excess consummation of bitter melon and excessive drinking of Asosi Tea every day during a very long period can enhance the toxicity or reduce the therapeutic effect of other drugs₁.

### R. STORAGE AND HANDLING

Dried Asosi leaves can be stored at any room temperature. Asosi Tea can be preserved in refrigerator until it reheats to get warm it to drink. Fresh Asosi must be dried in air to prevent mold.

### S. DANGER (HIGHLY IMPORTANT SAFETY INSTRUCTION ON THE HEAT PROCESS)

It is critical to take extreme precaution to reduce the risk of burns or fire. (YouTube Video might be produced to help some patients). Before taking the heat process, please lower the temperature of the stove on 5 or 6 to avoid burning in your face. Take extreme precaution to prevent the towel to catch fire. Clothing can stay or remove on patient when taking the heat process. Please don't use the specific heat process or Thermophore Heater on enfant (0-2 years) instead use a warm towel on the toddler front. Please don't use any hot towel on any enfants. It is very important to monitor kid, mental health and immune-compromised patients for safety concerns. In addition, do not drink hot Asosi Tea with lime. Don't give enfant or kid warm Asosi Tea with lime+honey (Asosi Tea at room temperature is required for enfant and kid).

### T. TOXICOLOGY

Cerasee demonstrated no toxicity during trials. It is also considered safe even for kids at moderate consumption of Asosi Tea. A late pregnant woman and 3-year-old drank Asosi tea to detox their body or to boost their immune system. They had no side effects or problem. However, taking Asosi Tea twice a week will enhance human immune system and increase fertility in woman. However, excessive used of Asosi Tea may increase toxicity or reduce other drugs therapeutic.

### U. INTERACTION WITH OTHER DRUGS

There are no data available on the presence of Cerasee Tea with lime with other drugs. However, Asosi Tea with lime may reduce the therapeutic effect of other drugs₁.

1. A method of taking ye-COVIDICIN Alternative Medicine treatment, comprising: a combination of heat process which is facial steaming by taking vapor directly from your front head for 5-15 minutes on boiling water on the stove for adult or enfant may or may not use warm towel compress smoothly in head and chest and kid may or may not use Thermophore or warm towel for 4-20 minutes (See Thermophore Specification); momordicilin extract from Cesasee or Asosi Leaves Tea with lime or lemon as a blood glucose, natural detox and antiviral agent; and without or with any antibiotic or antiviral if not allergic to speed up recovery.

2. Another method of taking ye-COVIDICIN Alternative Medicine for longer recovery, comprising: wherein the same heat process in claim 1; and any antibiotic if not allergic.
